(19) United States
(12) Patent Application Publication
(10) Pub. No.: US 2009/0012598 A1
(43) Pub. Date: Jan. 8, 2009

(54) METHOD OF FABRICATING AN IMPLANTABLE MEDICAL DEVICE BY DEFORMATION OF A TUBE OR A SHEET

(76) Inventors: ANTHONY J. ABBATE, Santa Clara, CA (US); David C. Gale, San Jose, CA (US); Klaus Kleine, Los Gatos, CA (US); Svava Maria Atladottir, San Francisco, CA (US)

Correspondence Address: SQUIRE, SANDERS & DEMPSEY LLP, 1 MARITIME PLAZA, SUITE 300, SAN FRANCISCO, CA, 94111, US

(21) Appl. No.: 12/169,588

(22) Filed: Jul. 8, 2008

Related U.S. Application Data
(63) Continuation of application No. 10/882,136, filed on Jun. 29, 2004, now abandoned.

(51) Int. Cl. A61F 2/06; B23P 13/04

(52) U.S. Cl. 623/1.15; 29/557; 623/1.16

(57) ABSTRACT

A method of fabricating an implantable medical device, such as a stent, from a plastically deformed tube or sheet is described herein. The implantable medical device may be an endoprosthesis such as a stent. The method may include elongating a tube longitudinally along a cylindrical axis of the tube. The method may further include forming a pattern that includes at least one strut on the elongated tube. A further embodiment may include stretching a sheet along an axis of stretching. The method may include forming a tube from the stretched sheet. The method may further include forming a pattern including at least one strut on the stretched sheet or the tube.

### CROSS-REFERENCE TO RELATED APPLICATIONS

This application is a continuation of application Ser. No. 10/882,136 filed on Jun. 29, 2004, which is incorporated by reference.

### BACKGROUND OF THE INVENTION

1. Field of the Invention

This invention relates to a method of fabricating implantable medical devices such as stents.

2. Description of the State of the Art

This invention relates to radially expandable endoprostheses which are adapted to be implanted in a bodily lumen. An “endoprosthesis” corresponds to an artificial device that is placed inside the body. A “lumen” refers to a cavity of a tubular organ such as a blood vessel. A stent is an example of these endoprostheses. Stents are generally cylindrically-shaped devices which function to hold open and sometimes expand a segment of a blood vessel or other anatomical lumen such as uninary tracts and bile ducts. Stents are often used in the treatment of atherosclerotic stenosis in blood vessels. “Stenosis” refers to a narrowing or constriction of the diameter of a bodily passage or orifice. In such treatments, stents reinforce body vessels and prevent restenosis following angioplasty in the vascular system. “Restenosis” refers to the reoccurrence of stenosis in a blood vessel or heart valve after it has been treated (as by balloon angioplasty or valvuloplasty) with apparent success.

A treatment involving a stent involves both delivery and deployment of the stent. “Delivery” refers to introducing and transporting the stent through a bodily lumen to a region requiring treatment. “Deployment” corresponds to the expanding of the stent within the lumen at the treatment region. Delivery and deployment of a stent are accomplished by positioning the stent about one end of a catheter, inserting the end of the catheter through the skin into a bodily lumen, advancing the catheter in the bodily lumen to a desired treatment location, expanding the stent at the treatment location, and removing the catheter from the lumen. In the case of a balloon expandable stent, the stent is mounted about a balloon disposed on the catheter. The stent is then expanded by inflating the balloon. The balloon may then be deflated and the catheter withdrawn. In the case of a self-expanding stent, the stent may be held in place on the catheter via a retractable sheath. When the stent is in a desired bodily location, the sheath may be withdrawn allowing the stent to self-expand.

Stents have been made of many materials such as metals and plastic, including biodegradable plastic materials. Stents have been formed from wire, tube stock, etc. Stents have also been made from sheets of material which are rolled into a cylindrical shape. The structure of a stent is typically composed of a pattern that allows the stent to be radially expandable. The pattern should be designed to maintain the longitudinal flexibility and radial rigidity required of the stent. Longitudinal flexibility facilitates delivery of the stent and radial rigidity is needed to hold open a bodily lumen. Since a stent is placed under a high degree of stress, particularly during deployment, it may be useful to employ methods of enhancing the mechanical properties of materials that make up a stent.

A number of techniques have been suggested for the fabrication of stents from sheets and tubes. One such technique involves laser cutting or etching a pattern onto a material. Laser cutting may be performed on a sheet of material which is then rolled into a tube. Alternatively, a desired pattern may be etched directly onto a tube. Other techniques involve cutting a desired pattern into a sheet or a tube via chemical etching or electrical discharge machining. Laser cutting of stents has been described in a number of publications including U.S. Pat. No. 5,780,807 to Saunders, U.S. Pat. No. 5,922,005 to Richter and U.S. Pat. No. 5,906,759 to Richter.

### SUMMARY OF THE INVENTION

The present invention is directed to a method for fabricating an implantable medical device, such as a stent from a tube, with desirable mechanical properties. A method of fabricating an implantable medical device may include elongating a tube along a cylindrical axis of the tube. The method may further include forming a pattern including at least one strut on the elongated tube.

A further embodiment of a method of fabricating an implantable medical device may include stretching a sheet along an axis. The method may include forming a tube from the stretched sheet. The method may further include forming a pattern including at least one strut on the stretched sheet and/or the tube.

### BRIEF DESCRIPTION OF THE DRAWINGS

FIG. 1 depicts a tube.

FIG. 2 depicts a planar view of an implantable medical device with a pattern.

FIG. 3 depicts a three-dimensional rendering of an implantable medical device with a pattern.

FIG. 4A depicts struts of an implantable medical device with a pattern.

FIG. 4B depicts an x-y coordinate plane.

FIG. 5 depicts elongation of a tube with a tensile force.

FIG. 6 depicts a radial cross-section of a tube.

FIG. 7 depicts an x-y coordinate plane.

FIG. 8A depicts stretching a sheet.

FIG. 8B depicts a tube.

FIG. 9 depicts a strut of a pattern.

### DETAILED DESCRIPTION OF THE INVENTION

For the purposes of the present invention, the following terms and definitions apply:

“Delivery diameter” refers to a diameter at which a cylindrical or substantially cylindrical implantable medical device, such as a stent, is introduced into and transported through a bodily lumen.

“Deployment diameter” refers to a diameter at which a cylindrical or substantially cylindrical implantable medical device, such as a stent, is expanded within a bodily lumen.

The “glass transition temperature,” T_{g}, is the temperature at which the amorphous domains of a polymer change from a brittle vitreous state to a solid deformable state at atmospheric pressure. In other words, the T_{g} corresponds to the temperature where the onset of segmental motion in the chains of the polymer occurs. When an amorphous or semicrystalline polymer is exposed to an increasing temperature, the coefficient of expansion and the heat capacity of the polymer both increase as the temperature is raised, indicating increased molecular motion. As the temperature is raised the actual molecular volume in the sample remains constant, and so a higher coefficient of expansion points to an increase in free volume associated with the system and therefore increased freedom for the molecules to move. The increasing heat capacity corresponds to an increase in heat dissipation through movement. T_{g} of a given polymer can be dependent on the heating rate and can be influenced by the thermal history of the polymer. Furthermore, the chemical structure of the polymer heavily influences the glass transition by affecting mobility.

The “melting temperature”, Tₘ, of a polymer is the highest temperature at which a crystal lattice in the polymer is stable.

“Stress” refers to force per unit area, as in the force acting through a small area within a plane. Stress can be divided into components normal and parallel to the plane, called normal stress and shear stress, respectively. Tensile stress and compressive stress are normal stresses.

The term “elastic deformation” refers to deformation of an object in which the applied stress is small enough so that the object retains its original dimensions or essentially its original dimensions once the stress is released. However, an elastically deformed polymer material may be prevented from returning to an undeformed state if the material is below the T_{g} of the polymer. Below T_{g}, energy barriers may inhibit or prevent molecular movement that allows deformation or bulk relaxation.

“Elastic limit” refers to the maximum stress that a material will withstand without permanent deformation.

The term “plastic deformation” refers to permanent deformation that occurs in a material under stress after elastic limits have been exceeded.

“Tensile strength” refers to the maximum stress in uniaxial tension in testing which a material will withstand prior to fracture. The ultimate tensile strength is calculated from the maximum load applied during the test divided by the original cross-sectional area.

“Tensile modulus” may be defined as the ratio of the stress or tensile force per unit area applied to a material divided by the amount of strain resulting from the applied force.

“Strain” refers to the amount of elongation, stretching, or compression that occurs in a material at a given stress or load.

“Elongation” may be defined as the increase in length which occurs when subjected to stress. It is typically expressed as a percentage of the original length.

The term “implantable medical device” is intended to include self-expandable stents, balloon-expandable stents, stent-grafts, and grafts. The structural pattern of the device can be of virtually any design. The device can also be made partially or completely from a biodegradable, bioabsorbable, or biostable polymer. The polymer may be purified. An implantable medical device may also be made of a metallic or ceramic material.

Polymers can be biostable, bioabsorbable, biodegradable, or bioerodable. Biostable refers to polymers that are not biodegradable. The terms biodegradable, bioabsorbable, and bioerodable are used interchangeably and refer to polymers that are capable of being completely degraded and/or eroded when exposed to bodily fluids such as blood and can be gradually resorbed, absorbed and/or eliminated by the body. The processes of breaking down and eventual absorption and elimination of the polymer can be caused by, for example, hydrolysis, metabolic processes, bulk or surface erosion, and the like. For coating applications, it is understood that after the process of degradation, erosion, absorption, and/or resorption has been completed, no polymer will remain on the device. In some embodiments, very negligible traces or residue may be left behind. For stents made from a biodegradable polymer, the stent is intended to remain in the body for a duration of time until its intended function of, for example, maintaining vascular patency and/or drug delivery is accomplished.

Representative examples of polymers that may be used to fabricate an implantable medical device using the methods disclosed herein include poly(N-acetylglucosamine) (Chitin), Chitoson, poly(hydroxyvalerate), poly(lactide-co-glycolide), poly(hydroxybutyrate), poly(hydroxybutyrate-co-valerate), polyorthoester, polyanhydride, poly(glycolic acid), poly(glycolide), poly(L-lactic acid), poly(L-lactide), poly(D,L-lactic acid), poly(D,L-lactide), poly(caprolactone), poly(trimethylene carbonate), polyethylene amide, poly(glycolic acid-co-trimethylene carbonate), co-poly(ether-esters) (e.g. PEO/PLA), polyphosphazenes, biomolecules (such as fibrin, fibrinogen, cellulose, starch, collagen and hyaluronic acid), polyurethanes, silicones, polyesters, polyolefins, polyisobutylene and ethylene-alphaolefin copolymers, acrylic polymers and copolymers other than polyacrylates, vinyl halide polymers and copolymers (such as polyvinyl chloride), polyvinyl ethers (such as polyvinyl methyl ether), polyvinylidene halides (such as polyvinylidene chloride), polyacrylonitrile, polyvinyl ketones, polyvinyl aromatics (such as polystyrene), polyvinyl esters (such as polyvinyl acetate), acrylonitrile-styrene copolymers, ABS resins, polyamides (such as Nylon 66 and polycaprolactam), polycarbonates, polyoxymethylenes, polyimides, polyethers, polyurethanes, rayon, rayon-triacetate, cellulose, cellulose acetate, cellulose butyrate, cellulose acetate butyrate, cellophane, cellulose nitrate, cellulose propionate, cellulose ethers, and carboxymethyl cellulose. Additional representative examples of polymers that may be especially well suited for use in fabricating an implantable medical device according to the methods disclosed herein include ethylene vinyl alcohol copolymer (commonly known by the generic name EVOH or by the trade name EVAL), poly(butyl methacrylate), poly(vinylidene fluoride-co-hexafluororpropene) (e.g., SOLEF 21508, available from Solvay Solexis PVDF, Thorofare, N.J.), polyvinylidene fluoride (otherwise known as KYNAR, available from ATOFINA Chemicals, Philadelphia, Pa.), ethylene-vinyl acetate copolymers, and polyethylene glycol.

A non-polymer substrate of the device may be made of a metallic material or an alloy such as, but not limited to, cobalt chromium alloy (ELGILOY), stainless steel (316L), high nitrogen stainless steel, e.g., BIODUR 108, cobalt chrome alloy L-605, “MP35N,” “MP20N,” ELASTINITE (Nitinol), tantalum, nickel-titanium alloy, platinum-iridium alloy, gold, magnesium, or combinations thereof. “MP35N” and “MP20N” are trade names for alloys of cobalt, nickel, chromium and molybdenum available from Standard Press Steel Co., Jenkintown, Pa. “MP35N” consists of 35% cobalt, 35% nickel, 20% chromium, and 10% molybdenum. “MP20N” consists of 50% cobalt, 20% nickel, 20% chromium, and 10% molybdenum.

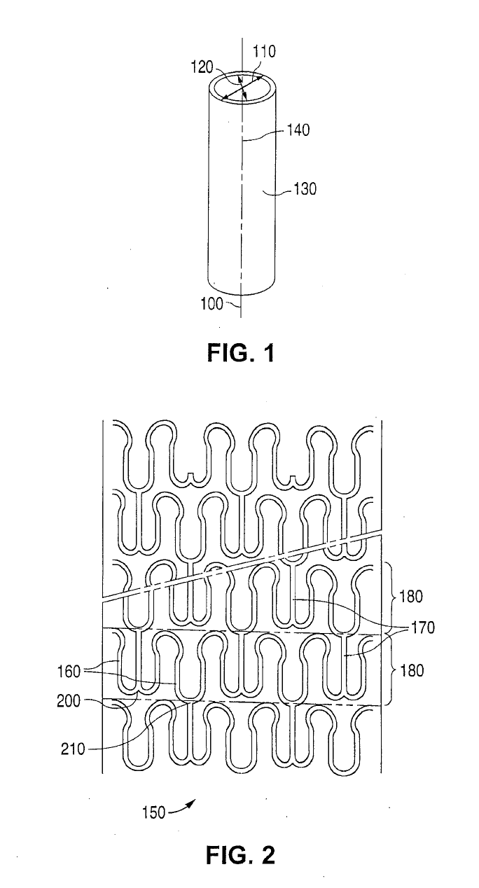

Some embodiments may include fabricating the implantable medical device from a conduit or tube. A pattern may be cut or etched in the tube. An embodiment may also include fabricating a device from a sheet that may be rolled into a tube and bonded. A pattern may be cut or etched in the sheet before or after rolling and bonding the sheet into a tube. The tube may be cylindrical or substantially cylindrical. For example, FIG. 1 depicts a tube 100. Tube 100 is cylindrical in shape with an outside diameter 110 and an inside diameter 120. FIG. 1 also depicts a surface 130 and a cylindrical axis 140 of tube 100. When referred to below, the “diameter” of the tube refers to the outside diameter of tube. In some embodiments, the diameter of the tube prior to fabrication of the implantable medical device may be between about 0.1 mm and about 30 mm. In other embodiments, the diameter of the tube prior to fabrication may be between about 1 mm and about 3 mm.

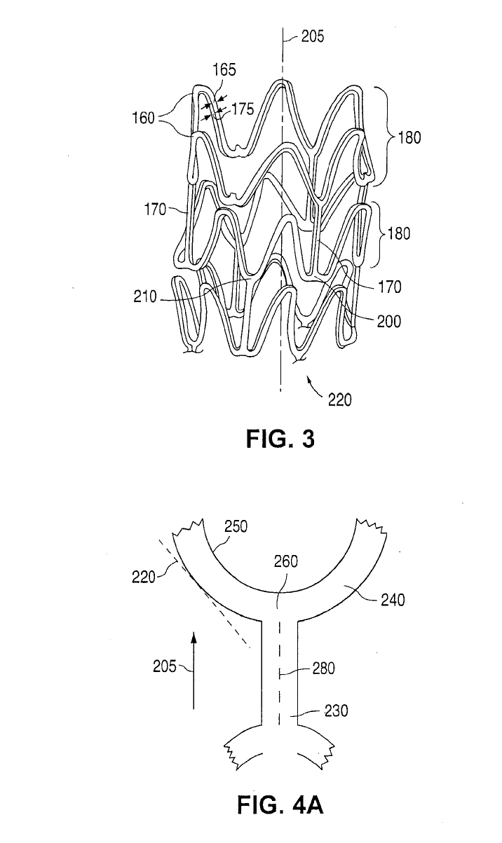

FIG. 2 depicts an example of a planar view of a pattern 150 formed using an etching or cutting process. Pattern 150 may include a number of interconnecting elements or struts 160 and 170. Pattern 150 also may include nodes 200 and 210, where a node refers to a portion of struts at which struts meet. In some embodiments, a subset of struts may form structural sections. For example, struts 160 form cylindrical rings 180. In addition, struts 170 connect cylindrical rings 180. FIG. 3 depicts a three-dimensional view of a cylindrical implantable medical device 220 which shows struts 160 and 170, cylindrical rings 180, and nodes 200 and 210. The latitudinal cross-section of the struts in device 220 is rectangular shaped. The dimensions of a strut may include a width 165 of the luminal and abluminal faces of a strut and a radial thickness 175. The cross-section of struts is not limited to what has illustrated and other cross-sectional shapes are applicable with the method of the invention. The implantable medical device may have a cylindrical axis 205. The final cut or etched out pattern should not be limited to what has been illustrated as other stent patterns are easily applicable with the method of the invention.

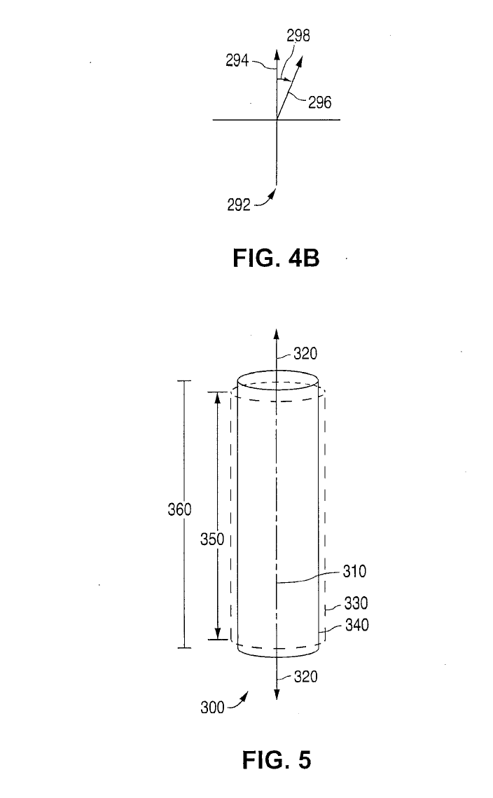

FIG. 4A depicts struts 230, 240, and 250 and node 260. The pattern on an implantable medical device, and therefore, the struts that make up the pattern may have a relative orientation with respect to the cylindrical axis or some other axis of the implantable medical device. Some other axis may be an axis along which the device material has been stretched. For example, cylindrical axis 205 of implantable medical device 220 in FIG. 3 relative to the struts is also depicted in FIG. 4A. An orientation of a strut relative to the cylindrical axis or some other axis of the implantable medical device may be defined by an angle of an axis of the strut with respect to the cylindrical axis or the some other axis of the implantable medical device. In one embodiment, an axis of a strut may be defined relative to an axis of connection of the strut to neighboring struts, which may be a longitudinal axis of a straight or substantially straight strut. For example, a straight or substantially straight strut such as strut 230 may have an axis 280. The axis of a curved strut may be defined by a tangent to the strut at a selected point. For example strut 250 may have an axis 290.

In addition, the relative orientation of an axis of a strut is illustrated in FIG. 4B. FIG. 4B illustrates the relative orientation of an axis of a strut relative to the cylindrical axis or some other axis of an implantable medical device using an x-y coordinate plane 292. An axis 294 represents the orientation of the cylindrical axis or some other axis of an implantable medical device. An axis 296 represents the orientation of an axis of a strut. An angle 298 depicts the relative orientation between the cylindrical axis or some other axis of the implantable medical device and the axis of the strut. When angle 298 is 0°, the axis of the strut is parallel to the cylindrical axis or some other axis of the implantable medical device. Similarly, when angle 298 is 90°, the axis of the strut is perpendicular to the cylindrical axis or some other axis of the implantable medical device.

It may be advantageous to modify the mechanical properties of a tube or sheet prior to forming a pattern. The mechanical properties may be modified to obtain improved strength, for example, of struts, and hence the implantable medical device. For example, the mechanical properties of a material may be modified by applying stress to the material. Modification of the mechanical properties of a material, such as a polymer, may be facilitated by applying heat as well as stress. It is believed that application of tensile stress to a polymer may alter the molecular geometry of the polymer. Rearrangement of polymer chains may take place when a polymer is stressed and consequently deformed. The polymer chains may become oriented along the direction of the applied stress which results in an oriented structure. A polymer stressed beyond its elastic limit generally retains its stressed configuration and corresponding molecular geometry when stress is removed.

The stressed polymer material may have a higher tensile strength along the direction of the applied stress. In addition, the modulus of a stressed material along the direction of applied stress may be increased which may correspond to lower elongation with applied stress. A plastically deformed material tends to retain its elongated configuration once the deforming stress is removed. Stress applied to a plastically deformed material subsequent to the initial deforming stress tends not to cause further elongation of the material unless the applied stress is greater than the initial stress level that caused the elongation. Therefore, the behavior of a plastically deformed material may be more predictable within a range of stress less than the initial or maximum stress applied that caused the plastic deformation.

A method of fabricating an implantable medical device may include elongating a tube longitudinally along a cylindrical axis of the tube. The tube may be elongated by application of a tensile force. In certain embodiments, the tube may be composed of a polymer material. In other embodiments, the tube may be metallic. In some embodiments, the tube may be elongated beyond the elastic limit of a tube material resulting in plastic deformation of the tube. In other embodiments, the tube may be elongated elastically. The method may further include forming a pattern that includes at least one strut on the elongated tube. In some embodiments, a tensile property of a strut on the elongated tube along the cylindrical axis of the elongated tube may be greater than the tensile property of an equivalent strut on an equivalent non-elongated tube along a cylindrical axis of the equivalent non-elongated tube. In an embodiment, a tensile property may include a tensile strength, tensile modulus, and/or resistance to elongation with applied stress.

In an embodiment, the equivalent non-elongated tube has the same dimensions and is composed of the same material as the elongated tube. The dimensions may include the length, diameter, and thickness of a tube. Furthermore, the dimensions of the strut on the elongated tube are equal to dimensions of the equivalent strut on the equivalent non-elongated tube. Also, the parameters characterizing a shape of the strut on the elongated tube are equal to parameters characterizing a shape of the equivalent strut on the equivalent non-elongated tube. In addition, an orientation of the axis of the strut on the elongated tube relative to the cylindrical axis of the elongated tube is equal to an orientation of the axis of the equivalent strut on the equivalent non-elongated tube relative to the cylindrical axis of the equivalent non-elongated tube.

In some embodiments, forming a pattern on the elongated tube may include laser cutting a pattern on the elongated tube. Representative examples of lasers that may be used include excimer, carbon dioxide, and YAG lasers. In other embodiments, chemical etching may be used to form a pattern on the elongated tube. It is desirable to use a laser cutting technique which minimizes material affected by the heat of the laser. Heat from the laser may tend to melt at least a portion of tube material in the heat affected zone. The alignment induced by the deformation may then be dissipated in the melted portion. The increase in a tensile property may also be reduced or substantially eliminated. In some embodiments, a pattern may be formed by chemical etching.

In an embodiment, the tensile force may be selected to achieve a desired degree of elongation. The tensile force may be constant or variable with time. In an embodiment, the tensile force may be configured to cause a constant rate of elongation of the tube. Elongating the tube may be performed with machines obtained, for example, from Instron Corporation of Canton, Mass.

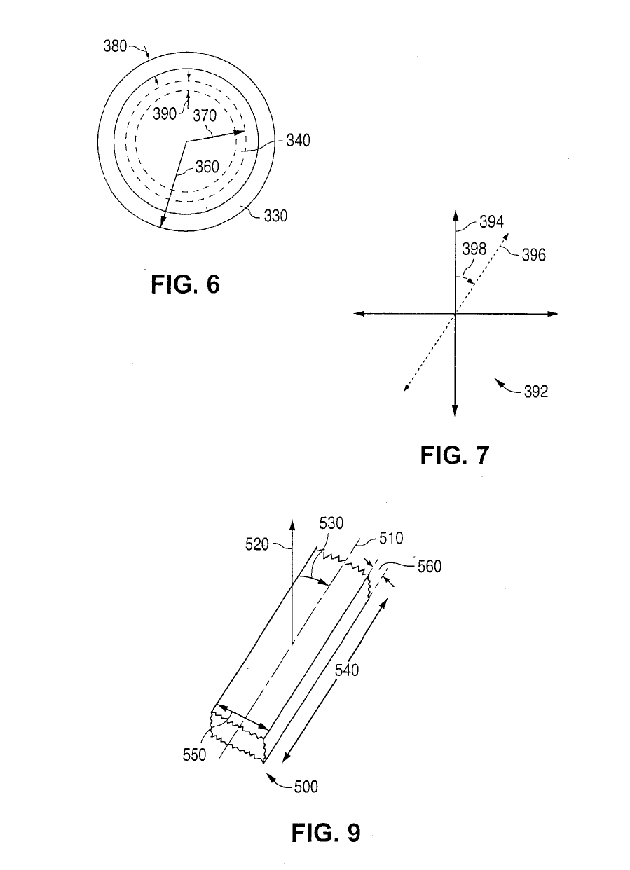

FIG. 5 depicts elongation of a cylindrical tube 300 with a tensile force 320. Cylindrical tube 300 may be elongated along a cylindrical axis 310 of tube 300 with tensile force 320. Tensile force 320 may cause tube 300 to elongate along its axis 310. A non-elongated tube 330 depicts the configuration of tube 300 prior to elongation and an elongated tube 340 depicts the configuration tube of 300 subsequent to elongation. A length 350 of tube 300 prior to elongation and a length 360 of tube 300 subsequent to elongation are depicted. In some embodiments, tensile force 320 and the resulting elongation of tube 300 may cause a decrease in a radius and thickness of tube 300. FIG. 6 depicts radial cross-sections of tube 330 with a radius 360 and tube 340 with a radius 370. In addition, a thickness 380 of tube 330 decreases to a thickness 390 of tube 340.

Another embodiment of a method of fabricating an implantable medical device may include stretching a sheet along an axis of stretching. The sheet may be stretched by application of a tensile force. In certain embodiments, the sheet may be composed of a polymer material. In other embodiments, the sheet may be metallic. In some embodiments, the sheet may be stretched beyond the elastic limit of a sheet material resulting in plastic deformation of the sheet along the axis of stretching. In other embodiments, the sheet may be stretched elastically. The method may also include forming a tube from the stretched sheet. The tube may be formed with a cylindrical axis of the tube parallel, perpendicular, or at an angle between parallel and perpendicular to the axis of stretching. Certain embodiments may further include forming a pattern that includes at least one strut on the stretched sheet and/or the tube.

In some embodiments, a tensile property of a strut on the tube along the axis of stretching may be greater than the tensile property of an equivalent strut on an equivalent tube formed from an unstretched sheet along the axis of stretching. In an embodiment, a tensile property may include a tensile strength, tensile modulus, and/or resistance to elongation with applied stress. In some embodiments, forming a pattern on the stretched sheet and/or the tube may include laser cutting or chemical etching.

In an embodiment, the equivalent tube has the same dimensions and is composed of the same material as the tube formed from the stretched sheet. The dimensions may include the length, diameter, and thickness of a tube. Furthermore, the dimensions of the strut on the tube formed from the stretched sheet are equal to dimensions of the equivalent strut on the equivalent tube. Dimensions of a strut may include length and latitudinal cross-sectional parameters. Latitudinal cross-sectional parameters may include abluminal width, luminal width, and radial thickness. Also, parameters characterizing a shape of the strut on the tube formed from the stretched sheet are equal to parameters characterizing a shape of the equivalent strut on the equivalent tube. In addition, the orientation of the axis of the strut on the tube formed from the stretched sheet relative to the axis of stretching of the tube formed from the stretched sheet is equal to an orientation of the axis of the equivalent strut on the equivalent tube relative to the axis of stretching.

Additional embodiments may include stretching the sheet along a second axis of stretching. The tube may be formed with a cylindrical axis of the tube parallel, perpendicular, or at an angle between parallel and perpendicular to the second axis of stretching.

In one embodiment, a sheet may be stretched along an axis using a tenter. In a tenter, stretching may be performed inside of a box. The temperature inside of the box may be controlled. Inside of the box, a sheet may be grasped on either side by tenterhooks that exert a tensile force or drawing tension along at least one axis.

In an embodiment, the tensile force for stretching a sheet may be selected to achieve a desired degree of stretching. The tensile force may be constant or variable with time. In an embodiment, the tensile force may be configured to cause a constant rate of stretching.

In certain embodiments, a tube may be formed from a sheet by rolling a sheet into a cylindrical shape. The sheet may then be bonded with a suitable adhesive at the opposing edges of the sheet that are parallel or substantially parallel to a cylindrical axis. The sheet may be cut so that the formed tube is a desired diameter.

FIG. 7 depicts an x-y coordinate plane 392 for illustrating the relationship between an axis of stretching 394 and a cylindrical axis 396 of a tube formed from a sheet. For example, a tube may be formed from a stretched sheet with an orientation 398.

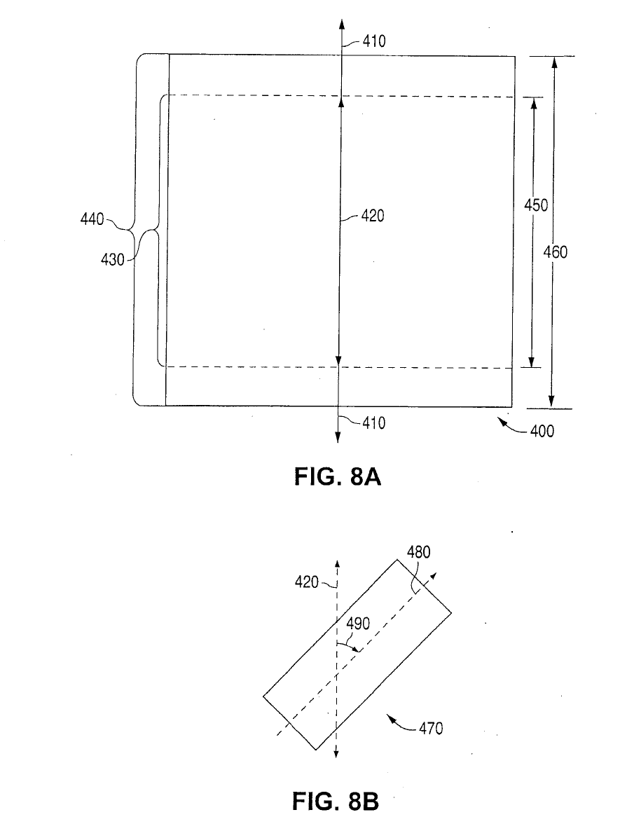

FIG. 8A depicts stretching of a sheet 400 with a tensile force 410. Sheet 400 may be stretched or elongated along an axis 420 parallel to tensile force 410. Tensile force 410 may cause sheet 400 to stretch along axis of stretching 420. An unstretched sheet 430 depicts the configuration of sheet 400 prior to stretching and a stretched sheet 440 depicts the configuration of sheet 400 subsequent to stretching. A length 450 of sheet 430 and a length 460 of sheet 440 are depicted. In some embodiments, tensile force 410 and the resultant stretching of sheet 400 may cause a decrease in a width and/or thickness of sheet 400. FIG. 8B depicts a projection of a tube 470 formed from sheet 400 after stretching. Tube 470 has a cylindrical axis 480 which is at an orientation 490 relative to axis of stretching 420.

As discussed above, a tensile property of an elongated tube or a tube formed from a stretched sheet may be greater, which may be more desirable, along the axis of the elongation or stretching than a tube that has not been elongated or not formed from a stretched sheet, respectively. Similarly, a tensile property of struts that are part of a pattern on an elongated tube or a tube formed from a stretched sheet may be greater or more desirable than equivalent struts on a tube that was not elongated or a tube formed from an unstretched sheet, respectively. For example, FIG. 9 depicts a strut 500, with an axis 510 that may be on an elongated tube or a tube formed from a stretched sheet. Strut 500 has dimensions of a length 540, a width 550, and a radial thickness 560. An axis 520 depicts an axis of elongation or stretching. An orientation 530 corresponds to the relative orientation of axis 510 of the strut with axis 520. A strut equivalent to strut 500 may be on an equivalent non-elongated tube or an equivalent tube formed from an unstretched sheet. Strut 500 on the elongated tube or the tube formed from a stretched sheet may tend to have greater or more desirable tensile properties along axis 520 than an equivalent strut on an equivalent non-elongated tube or equivalent tube formed from an unstretched sheet along the same axis. An equivalent strut may have the same angle of orientation 530 and the same dimensions 540, 550, and 560 as the strut on the elongated tube or the tube formed from a stretched sheet. Also, an equivalent strut has the same shape. As illustrated in FIGS. 2-4, a strut may have curved portions. The curved portions may be characterized by a parameter such as the radius of curvature of a curved portion of a strut.

In certain embodiments of the method, a tensile property along an axis of the strut on the elongated tube or tube formed from a stretched sheet may be greater or more desirable than the tensile property along the axis of the equivalent strut on the equivalent non-elongated tube or an equivalent tube formed from an unstretched sheet, respectively. For example, a tensile property along axis 510 in FIG. 9 of the strut on the elongated tube may be greater than the tensile property along the same axis of the equivalent strut formed on an equivalent non-elongated tube. In general, the increase in tensile strength of a material due to elongation or stretching tends to be greatest along an axis parallel to the axis of elongation or stretching and least along a an axis perpendicular to the elongation or stretching. Therefore, the smaller the angle of orientation 530, the greater the improvement in a tensile property between a strut on an elongated tube or tube formed from a stretched sheet and an equivalent strut on an equivalent non-elongated tube or tube formed from an unstretched sheet, respectively.

Moreover, it may be desirable to form a pattern to obtain a selected value or range of a tensile property of the strut on the elongated tube or tube formed from a stretched sheet. For instance, it may be desirable to form a pattern that minimizes orientation 530 in FIG. 9 of at least one or most of the struts or portions of some of the struts close to zero. In some embodiments, the pattern can be selectively formed so as to maximize tensile properties in at least one strut or designated segments of the stent. Generally, the method may include forming a pattern to obtain a selected orientation of an axis of the strut relative to the axis of elongation or stretching.

Additionally, a tube may be formed to obtain a selected orientation of the cylindrical axis of the tube relative to the axis of stretching. Similarly, the tube may be formed to obtain a selected orientation of an axis of a strut or struts relative to the axis of stretching. A sheet may be rolled and bonded along any desired axis relative to the stretching. As a result, mechanical properties may be modified along any axis with respect to the cylindrical axis or axis of a strut or struts. Therefore, a tube may be formed from a sheet that has a desired relative orientation of the axis of stretching to the axis of a strut or struts in a pattern. The struts may be formed on the sheet or formed on a tube formed from a sheet.

As discussed above, it may be advantageous to elongate tube 300 in FIG. 5 beyond its elastic limit. As described above, the tube or sheet may tend to retain its stressed configuration when it is elongated or stretched beyond a yield point. The plastically deformed tube or tube formed from a plastically deformed sheet may have more favorable mechanical behavior, such as tensile strength, modulus, and resistance to elongation or stretching with applied stress along an axis of elongation or stretching. Therefore, some embodiments of the method may include selecting the tensile force to elongate or stretch the tube or sheet greater than the elastic limit of the tube or sheet material. In addition, the tensile force may be selected to obtain a desired range or value of a tensile property of the elongated tube or tube formed from the stretched sheet and/or struts.

For polymer tubes or sheets, it may be desirable to elongate the tube or stretch a sheet at a temperature equal to or greater than the T_{g} of the polymer and less than or equal to the melting temperature of the polymer, Tₘ. Above Tₘ the shape of the implantable medical device is unstable since the polymer exists as a polymer melt or in a molten state. In other embodiments, a tube may be elongated or a sheet stretched at a temperature below T_{g}.

Moreover, in certain embodiments, a method of fabricating a tube may further include applying heat to the tube or sheet. Application of heat with stress may facilitate elongation or stretching of a polymer under tension, and hence, modification of the tensile properties of the polymer. It is believed that application of heat increases polymer chain mobility which facilitates deformation. Polymer chain mobility in solid polymer is very limited due to strong chain interaction and/or crystallization. In a solid-state deformation process, the interaction between chains restricts the achievable orientation and creates a high level of stretching tension. The application of heat may be prior to, contemporaneous with, and/or subsequent to elongating a tube. Similarly, the application of heat may be prior to, contemporaneous with, and/or subsequent to stretching a sheet.

In some embodiments, heat may be applied by contacting the tube or sheet with a fluid above ambient temperature. An ambient temperature may be about 25° C. For example, the tube or sheet may be immersed in a liquid and/or contacted with a stream of gas. The tube or sheet may be translated through the liquid and/or the stream of gas. In addition, the tube or sheet may be heated through contact and/or close proximity to a heated object. In another embodiment, heat may be applied by using an oven. In other embodiments, the expansion of the tube or stretching of the sheet may be performed satisfactorily without application of heat. In particular, this may be the case with polymers that have T_{g}'s below an ambient temperature.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

1-20. (canceled)

21-58. (canceled)

59. A method of manufacturing an implantable medical device, comprising:
stretching a sheet along an axis of stretching;
forming a tube from the stretched sheet; and
forming a pattern comprising at least one strut on the stretched sheet and/or the tube.

60. The method of claim 59, wherein a cylindrical axis of the tube is parallel, perpendicular, or at an angle between parallel and perpendicular to the axis of stretching.

61. The method of claim 59, further comprising stretching the sheet along a second axis of stretching.

62. The method of claim 61, wherein a cylindrical axis of the tube is parallel, perpendicular, or at an angle between parallel and perpendicular to the second axis of stretching.

63. The method of claim 59, wherein the implantable medical device is a stent.

64. The method of claim 59, wherein the sheet comprises a polymer.

65. The method of claim 59, wherein the sheet comprises a bioabsorbable polymer.

66. The method of claim 59, wherein the tube is cylindrical or substantially cylindrical.

67. The method of claim 59, wherein the sheet comprises a metal.

68. The method of claim 59, wherein a diameter of the tube formed from the stretched sheet comprises approximately a delivery diameter of an implantable medical device.

69. The method of claim 59, wherein a tensile property of a strut on the tube along the axis of stretching is greater than the tensile property of an equivalent strut on an equivalent tube formed from an unstretched sheet along the axis of stretching.

70. The method of claim 69, wherein dimensions of the tube formed from the stretched sheet are equal to dimensions of the equivalent tube.

71. The method of claim 69, wherein dimensions of the strut on the tube formed from the stretched sheet are equal to dimensions of the equivalent strut on the equivalent tube.

72. The method of claim 69, wherein parameters characterizing a shape of the strut on the tube formed from the stretched sheet are equal to parameters characterizing a shape of the equivalent strut on the equivalent tube.

73. The method of claim 69, wherein the tensile property along an axis of the strut on the tube formed from the stretched sheet is greater than the tensile property along the axis of the equivalent strut on the equivalent tube.

74. The method of claim 73, wherein an orientation of the axis of the strut on the tube formed from the stretched sheet relative to the axis of stretching of the tube formed from the stretched sheet is equal to an orientation of the axis of the equivalent strut on the equivalent tube relative to the axis of stretching.

75. The method of claim 59, wherein the pattern is formed to obtain a selected value or range of a tensile property of at least one strut on the tube formed from the stretched sheet.

76. The method of claim 59, wherein the pattern is formed to obtain a selected orientation of an axis of at least one strut relative to the axis of stretching.

77. The method of claim 59, wherein the pattern is formed to maximize a tensile property along an axis of at least one strut.

78. The method of claim 59, wherein the tube is formed to obtain a selected orientation of the cylindrical axis of the tube relative to the axis of stretching.

79. The method of claim 59, wherein the tube is formed to obtain a selected orientation of an axis of at least one strut relative to the axis of stretching.

80. The method of claim 59, wherein the tube is formed to maximize a tensile property along an axis of at least one strut.

81. The method of claim 59, wherein the tensile property comprises a tensile strength.

82. The method of claim 59, wherein the tensile property comprises a tensile modulus.

83. The method of claim 59, wherein the tensile property comprises resistance to elongation with applied stress.

84. The method of claim 59, wherein forming the pattern comprises laser cutting the pattern.

85. The method of claim 59, wherein the sheet comprises a polymer, and wherein the stretching is performed at a temperature greater than or equal to the glass transition temperature of the polymer and less than or equal to the melting temperature of the polymer.

86. The method of claim 59, wherein the sheet comprises a polymer, and wherein the stretching is performed at a temperature less than the glass transition temperature of the polymer.

87. The method of claim 59, wherein the sheet is stretched plastically.

88. The method of claim 59, wherein the sheet is stretched by application of a tensile force.

89. The method of claim 59, further comprising applying heat to the tube.

90. The method of claim 89, wherein the application of heat is prior to, contemporaneous with, and/or subsequent to the stretching.
